(19) United States
(12) Patent Application Publication
(10) Pub. No.: US 2013/0018279 A1
(43) Pub. Date: Jan. 17, 2013

(54) "blood sample collection apparatus and kits"

(71) Applicants: James Plante, La Jolla, CA (US); Michael Nova, Del Mar, CA (US); David Becker, San Diego, CA (US); Luke Noaek, San Francisco, CA (US)

(72) Inventors: James Plante, La Jolla, CA (US); Michael Nova, Del Mar, CA (US); David Becker, San Diego, CA (US); Luke Noaek, San Francisco, CA (US)

(73) Assignee: Pathway Genomics

(21) Appl. No.: 12/584,153

(22) Filed: Sep. 1, 2009

(51) Int. Cl. A61B 5/151

(52) U.S. Cl. 600/583

(57) ABSTRACT

Blood sample collection apparatus and kits are devised as easy-to-use, reduced pain devices operable by persons not having medical training or other expertise or knowledge. A flexible strip having adhesives on two portions of a first side accommodate therebetween and absorbent pad and substrate with microstructures. The substrate includes microstructures arranged to gently penetrate or pierce skin and cause a small amount of blood to pass to the external surface of a user's flesh. An absorbent pad receives and absorbs a blood sample therein. The absorbent pad may additionally support a plurality of chemical agents which further advances mobility of blood in the collection system. These chemical agents may include anticoagulants and blood anticlotting agents to facilitate blood transfer from a donor to the absorbent pad. Additionally, these systems may also include a blood preservative to stabilize blood collected for an extended shipping and transfer period. These blood sample collection systems may further include a numbing agent to improve comfort of use.

### BACKGROUND OF THE INVENTION

1. Field

The following invention disclosure is generally concerned with blood sample collection apparatus and kits and specifically concerned with apparatus and kits which may be deployed and fully operated in home use environments by non-skilled users.

2. Prior Art

Modern medical analytical systems have matured to a state whereby inexpensive and ready access to sophisticated testing may be provided without inconvenient visits to doctor's offices, laboratories, medical clinics et cetera. In particular, blood metabolic analysis performed in an advanced modern laboratory only requires a patient to provide a very small quantity of blood as a sample. While drawing blood for metabolic testing remains largely a process taken up by medical specialists (e.g. nurses) with advanced training in medical procedures in a controlled environment such as a doctor's office or hospital with specialized expensive equipment, new health philosophies suggest omission of the doctor/hospital visit and replacement with a ‘direct-to-consumer’ process including submission of samples of biological matter such as blood by way of easy-to-use, self-contained sample collection systems which may be fully deployed directly by the consumer.

Presently, when a person desires analysis on blood metabolites, a sample of blood is drawn by a professional with specialized equipment (sterile collection and puncture equipment for example). A nurse will take blood by piercing the skin with a hypodermic needle, drawing blood into a suitable receptacle, removing the needle, dressing the puncture site, and conveying the sample to appropriate testing facility via inter-office medial material handling systems.

Although blood is most generally drawn by specialists, it nevertheless remains a common process in which a person may perform a self administered blood draw. One may puncture his own skin, collect a blood sample and transmit that to a testing facility without the aid and assistance of a professional trained in medical procedure. Of course this is well known to most diabetics who commonly perform similar operations daily. In most advanced systems, a microlance device, sometimes spring-loaded, is used by diabetic patients to pierce the skin where a small drop of blood can be collected (for example on a test strip having reactive agents thereon) at the surface of the tissue. The blood drop is thereafter subject to testing to determine the level of glucose in the blood.

While not strictly related to blood sample collection systems, a common bandage arranged as an inexpensive adhesive strip nevertheless has important characteristics associated with teachings presented herein. A Band-Aid® type disposable strip bandage is generally used to stop bleeding and protect a fresh wound from germs. Such bandage typically includes a plastic strip coupled with or having affixed thereto an absorbent pad. The plastic strip additionally includes adhesive on either side of the pad which permits the device to be attached and held to undamaged skin about the wound. When properly applied, a bandage as described will absorb blood and fluids and remove same from the wound. After some time, the wound heals and the bandage may be removed and discarded. In some highly functional versions, chemical additives may be additionally included to prevent runaway bacterial growth. An antibacterial agent may be incorporated with the absorbent pad whereby the wound is further protected from infection. Merely applying the bandage also applies the antibacterial agent. Although bandages such as these operate to draw blood from tissue, they are in fact not suitable for blood sample collection functionality.

While systems and inventions of the art are designed to achieve particular goals and objectives, some of those being no less than remarkable, these inventions of the art have nevertheless include limitations which prevent their use in new ways now possible. Inventions of the art are not used and cannot be used to realize advantages and objectives of the teachings presented herefollowing.

### SUMMARY OF THE INVENTION

Comes now, James Plante, David Becker, Michael Nova, and Luke Nosek with inventions of a blood sample collection apparatus and kits including those having a skin piercing structure which may be applied to the skin surface by way of adhesives on a thin strip.

A blood sample collection system is arranged as a plastic strap having incorporated therewith a skin piercing element and an absorbent pad. When applied to the surface of a donors tissue, for example the arm, the piercing element compromises the integrity of the skin and causes blood to be drawn to the surface. Blood leaving the tissue comes into contact with the absorbent pad and is further drawn into and received there.

In most important versions, the piercing element is formed as a microstructure system of tiny needles or lances or barbs. These operate to get may pierce the skin without excessive pain. Similar to the manner in which a mosquito manages to pierce the skin a man-made microneedle can be inserted deep enough into the tissue to reach blood vessels while very being perceptible by the human being injected.

In versions where microneedles are used, the needles are useful for providing a conduit for blood to pass from a blood vessel to the absorbent pad. In special versions where barb type microstructures are formed, the barbs tend to advance the piercing element into the tissue to assure the device will reach a data sufficient to extract blood. Actually occurring motions tend to drive the barbs deeper into the tissue. In some versions, a mere lands die micro knife is sufficient to penetrate the skin and cause blood to be released to the absorbent pad.

In certain versions, the absorbent pad additionally has integrated therewith chemical agents to advance and bring about important related function. In a first version, an anticoagulant or anticlotting blood thinning chemical is used to assure blood will more numbing agent or pain relieving drug is added to alleviate discomfort which might otherwise be generated by the piercing element. In still further important versions, kits may be provided where special facility is arranged for containing and preserving a blood sample in a package configured for use with public and private delivery services.

In most favored versions, an indicator is provided to alert users to the condition whereby the absorbent pad has received a sufficient quantity of blood. A reactive agent provides in the shop on a surface viewable by the donor user.

### OBJECTIVES OF THE INVENTION

It is a primary object of the invention to provide new blood sample collection systems.

It is an object of the invention to provide blood sample collection systems operable by consumers without specialized medical training or experience.

It is a further object to provide blood collection systems compatible with the mails and private delivery services.

It is an object of the invention to provide blood collection systems which preserve a blood sample for a period between 1-14 days.

It is an objective to provide blood collection systems which operate to take a blood sample of sufficient size to support metabolic blood testing systems.

A better understanding can be had with reference to detailed description of preferred embodiments and with reference to appended drawings. Embodiments presented are particular ways to realize the invention and are not inclusive of all ways possible. Therefore, there may exist embodiments that do not deviate from the spirit and scope of this disclosure as set forth by appended claims, but do not appear here as specific examples. It will be appreciated that a great plurality of alternative versions are possible.

### BRIEF DESCRIPTION OF THE DRAWING FIGURES

These and other features, aspects, and advantages of the present inventions will become better understood with regard to the following description, appended claims and drawings where:

FIG. 1 is view of two opposing sides of an adhesive strip of these systems;

FIG. 2 is a side-view cross section of an blood sample collection system of this disclosure;

FIG. 3 is a close-up view of an important element—the absorbent pad and a spatial distribution of cooperating chemicals;

FIG. 4 is a perspective view of an example of integration between absorbent pad and a piercing element of preferred blood sample collection systems;

FIG. 5 illustrates in a side-view cross section another special supporting element;

FIG. 6 is very close-up view of a single microstructure of special nature and cooperating substrate;

FIG. 7 shows an alternative version of microstructure of particular importance; and

FIG. 8 shows in perspective kits of this invention and elements thereof in a cooperating relationship.

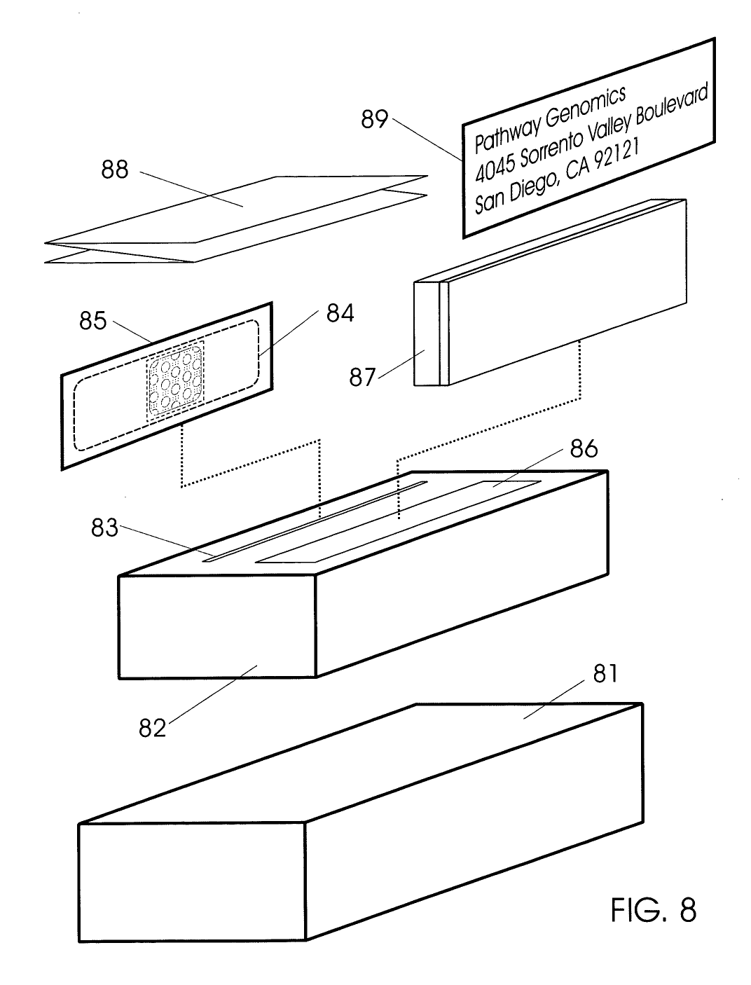

### PREFERRED EMBODIMENTS OF THE INVENTION

In accordance with each of preferred embodiments of the invention, blood sample collection apparatus and kits are provided. It will be appreciated that each of the embodiments described include an apparatus and/or kit and that the apparatus and/or kit of one preferred embodiment may be different than the apparatus or kit of another embodiment. Accordingly, limitations read into one example should not be carried forward and implicitly assumed to be part of any alternative example.

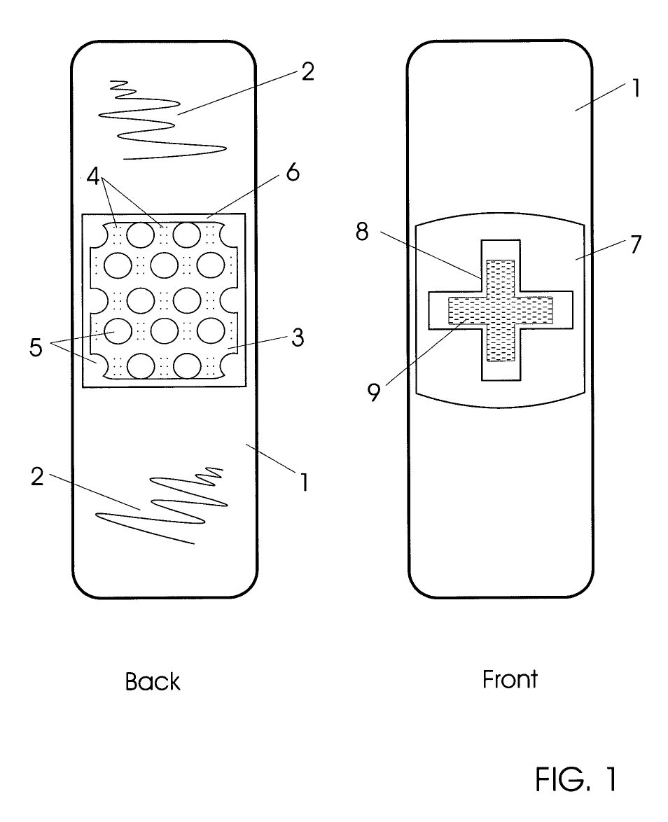

With reference to the drawing figures, and particularly to FIG. 1 which illustrates front and back view, of a first version of a blood sample collection apparatus of this invention in simple line drawing. A main body strip 1 element provides support and structure upon which other cooperating elements are coupled and/or integrated. In particular, adhesive matter 2 distributed about a large area portion of the strip surface on either side of center. Preferred adhesives include those compatible for use with human skin and more particularly, those which further include a ‘release’ capacity whereby a bond between skin and the strip may be decoupled without significant pain and discomfort or damage to tissue from where it was coupled prior to release.

A substrate 3 of rigid or semi-rigid material forms a base upon which microstructure elements 4 may be formed to protrude orthogonally from the plane of the substrate. In some important versions, the substrate may be embodied as a crystalline silicon wafer. In some versions, microstructures can be provided by crystal growth, etching and microlithography processes. In these versions, a base element upon which these microstructures are grown is preferably silicon as the semiconductor industry has developed many important capabilities in silicon wafer processing sciences. However, as the silicon wafer has little functional value after providing a crystal seed and mechanical support for microstructures, many alternatives are possible. Accordingly, it will be noticed that the primary function of a substrate described here is mechanical support for microstructures and many alternative materials might provide identical function.

In best versions of these devices, a substrate element includes a plurality of holes 5 provided therein and spatially distributed thereabout. These holes provide a pathway for blood transmission from a first side of the substrate to the other side. Blood incident upon a first side of the substrate may easily pass to the other side by way of the holes therein. Similarly, other chemical matter on one side of the substrate may easily pass to the other by natural migration. Accordingly, these substrates are prepared to permit fluids to easily and readily pass therethrough without regard to the material from which they are comprised.

Integrated with the substrate is an absorbent pad 6 of cloth or nonwoven matrix of fiber formed in sheets for example. A pad is provided to receive, absorb and store a blood sample.

In one important alternative version, an absorbent pad may be made from a sponge or sponge like element. A sponge made of synthetic material may be particularly attractive as it offers some additional functional value. In one case, an ‘elastomer’ sponge is effective for maintaining a blood sample in a dried state for extended periods of time. The sample may be rehydrated and removed from the sponge for testing. Upon receiving the sample at the absorbent pad made of elastomer sponge, the sample may be subject to natural drying or even accelerated drying via a desiccant. Important characteristics of absorbent pads taught here require they be absorbent, suitable for long term storage of biological matter, suitable for integration and bonding with a strip such as a polymer or plastic strip.

An additional feature and function of the pad elements provided here is to store and carry various chemical agents which are provided to aid in the functionality and objectives of these systems.

The absorbent pad may be affixed and coupled to the strip by a permanent glue adhesive whereby it cannot be separated therefrom without destructive results. The pad is best located at the strip's center region as shown in the diagram. While its shape is not critical, the purpose of the system suggests an oval or rectangular shape of aspect ratio approximately equal to one. As blood is released over a small area, it tends to naturally spread evenly in two dimensions and thus the pad provides better function where it is similarly symmetric.

A second side of the main body strip provides additional important function. As nonexperts of medical procedure are primary target users of devices taught herein, it is important to provide clear use guides and mechanisms. Among these is an indicator system arranged to provide a visual signal indicative of the state and condition of use with regard to receipt of a sufficient quantity of blood in the absorbent pad.

In one first version, an opaque cover element 7 has a shaped see-through window 8 integrated therein. Behind this window and cover element is a reactive agent or change medium 9 which yields a color change or other state change as a reaction to the presence of certain prescribed matter, for example blood. In one important example version of a reactive agent or change medium, a material which changes color in response to change in pH. When a chemical (e.g. blood) of prescribed pH is present, the agent changes from a first color to a second color, or from clear to opaque. The change is easily detected as a visual signal by a nonscientific or non-sophisticated user.

In another important version of an indicator system useful with these devices includes one based upon a change medium which changes its polarization state in the presence of blood. In this version the window is particularly configured to cooperate with the polarization state of the change medium to form this version of indicator system. The window may be arranged as a polarizer of one nature while the change medium operates (when blood is present) as a polarizer of cooperating nature. Together, they can produce a useful visual signal at the window.

In best versions of these indicator systems, the window is shaped with a periphery of special regard. To indicate ‘affirmative’ or ‘positive’ condition, a symbol such as a ‘check’ or ‘plus’ sign may be used as it is easily read and immediately imparts a confirmation of a desired condition without ambiguity. This makes it easier for nonexpert lay persons to interpret the signal provided.

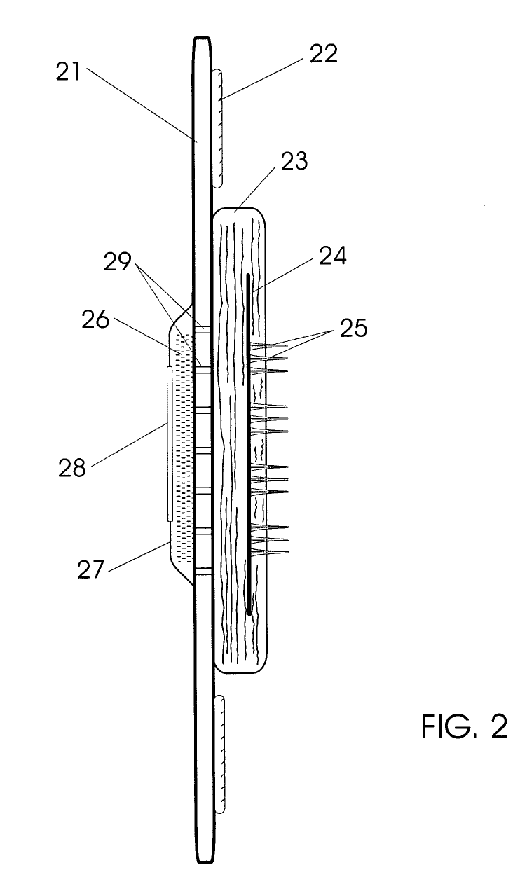

Greater understanding of these blood sample collection apparatus is realized in consideration of the side view diagram of FIG. 2 and detailed description here, where another important example version is presented. For clarity, the scale of the drawing in orthogonal dimensions is different. Scale has been expanded in the horizontal dimension by a factor of about three or more. It will be appreciated that these devices are specifically designed as a plurality of very thin strips. Diagrams of many thin strips may be difficult to understand. As such and in further view of complete clarity, the drawing of FIG. 2 is provided with very thin elements drawn with expanded scale.

A strip main body 21 portion has thereon at both ends adhesive material 22. Adhesives used in common strip type bandages work identically in these apparatus. Adhesive material applied to one side of the strip operates to form a temporary bond to a human skin. The bond, while durable may be released after a brief time by merely pulling the strip away from the site to which it is affixed.

An absorbent pad 23 may be formed in layers. In some versions, the pad is preferably formed of a plurality of layers which can be laid together one on top of another to form the pad. Natural or synthetic fibers can both be arranged to provide for these pads. It is a primary function that the pad operate to absorb and store blood samples therein. Accordingly, a fiber selected to improve this function improves the performance of the devices. Absorbent objects characterized as ‘sponge like’ are fully anticipated.

An absorbent pad may have integrated therewith substrate 24 upon which microstructures 25 are formed and mechanically supported to form a piercing element. A piercing element of these devices may have a plurality of individual microstructures each which may operate independently to pierce skin, however, for this nomenclature the entire substrate and collection of microstructures together is called ‘piercing element’. A piercing element of this type may be formed in a crystal growth process and fabricated separately before being incorporated with the absorbent pad.

In one alternative version, a substrate is first directly affixed to the strip and the absorbent pad is placed thereon. Microstructures may pass through material of the absorbent pad and be exposed on the other side. These versions may permit a simplification in manufacturing. The precise location of the piercing element substrate is not critical—however, it is preferred that it be near to the surface of the assembled device to permit shorter stronger microstructures to be used.

On an opposing side of the strip, an indicator system is comprised of a change medium 26, a cover element 27, and window 28. The strip may be further prepared with via holes 29 which permit a quantity of blood to pass from the absorbent pad into the change medium. Blood which passes from the absorbent pad, through the vias and into the change medium or ‘reactive agent’ causes a state change therein. The state change may provide a visual signal which can be viewed by a user through the window.

An absorbent pad of these systems includes primary functions including: to receive, store, and preserve a blood sample for an extended period of time, and further to carry, and in some cases deliver chemical agents to improve system functionality. With regard to chemicals included in some versions of these absorbent pads which improve functionality, these may include: preservatives, anticoagulants, numbing agents, among others.

A numbing agent is sometimes used to improve comfort to the user. Because these devices necessarily pierce a user's skin, normal use is sometimes accompanied by slight and minor pain. While certain skin piercing microstructures have been formed with the objective to minimize pain, their use does nevertheless sometimes cause slight pain in some more sensitive users. Accordingly, a numbing agent may be included as part of the system. Where the device is placed into contact with a user's skin, the numbing agent is conveyed to the user's tissue where it may be absorbed and operate to reduce any pain caused by further penetration of the microstructures.

A second functional chemical agent which may be integrated with the absorbent pad includes an anticlotting or anticoagulant. To improve the mobility of blood received in the pad, an anticoagulant or blood thinning agent can be used in certain versions of these blood sample collection apparatus. It is useful to make the pad more absorbent so that blood is quickly pulled away from the skin surface and moved deep into the pad layers. For this, and anticoagulant which mixes with blood as it arrives permits the blood to easily pass into the pad and move therewithin. Further, in versions deploying an indicator system, blood must have high enough mobility to traverse the entire depth of all layers to finally reach the reactive agent. For this reason, anticoagulants integrated with the pad further improve system functionality.

These systems are intended to serve a very unique distinct function not found in other blood sample collection systems. In particular, as these apparatus are primarily intended for home use by nonexperts or persons without special medical skills, it becomes necessary to provide both mechanical and functional aspects to support use by untrained persons. In addition, a conveyance or delivery mode of use is also supported. That is, after blood is received by the apparatus, the apparatus must store and hold the sample in good condition for an extended time whereby it may be conveyed from the consumer user to a testing facility i.e. between about one and five days. Accordingly, versions of these systems include special supporting packaging (described in details herefollowing) as well as chemical agents to preserve and stabilize a blood sample for extended periods. These might include antibacterial agents, blood stabilization chemistry, among others.

Because the function associated with each of these chemical agents is different, there is sometimes an advantage to providing them in a spatial distribution which improves their functionality. That is, it becomes preferable to provide the numbing agent at or near the exposed surface as it only is effective where it is passed to the user's tissue via the site to which the device is affixed. As such, these devices are best arranged with a high concentration of numbing agent nearest the exposed surface of the absorbent pad. In addition, while blood mobility is important throughout it is more important at the surface and internal structures where blood might tend to clot. Once blood is well distributed and reaches all the way to the reactive agent of the indicator system, mobility is no longer important. Accordingly, in some version a radial distribution where concentration decreases as a function of radius ‘r’ improves performance in this regard.

In another important spatial consideration, it is desirable to preserve blood distributed throughout the device. It is important to knock down bacterial growth everywhere. As such, a uniform distribution of preservative is desirable in highest performance versions. Accordingly, chemical agents included in these apparatus may include spatial distributions which are advantageous to the particular function associated with the chemical and its function.

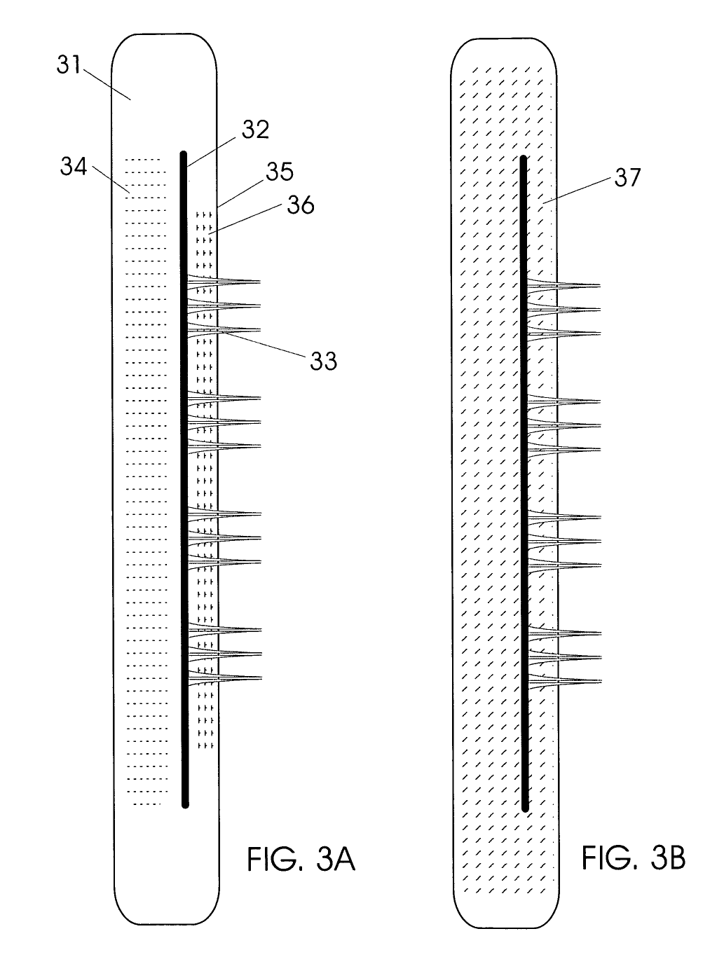

FIGS. 3A and 3B illustrate one example version of spatial distribution of chemical agents deployed in absorbent pads of these versions. Absorbent pad 31 has integrated therewith a substrate 32 upon which microstructures 33 are mechanically supported. A liquid blood thinning agent or anticoagulant 34 may be provided within the pad as shown to facilitate and increased blood mobility. In this way blood received at the absorbent pad surface will be encouraged to past towards the opposing surface of the pad. At and near the exposed surface 35 of the absorbent pad, a chemical numbing agent 36 may be distributed. In this way, once a pad comes into contact with a user's skin surface, the numbing agent tends to be transferred thereto. In a second view, FIG. 3B, of the same absorbent pad a chemical 37 is uniformly distributed. An antibacterial or other blood preserving agent is provided in all portions of the pad which may receive blood.

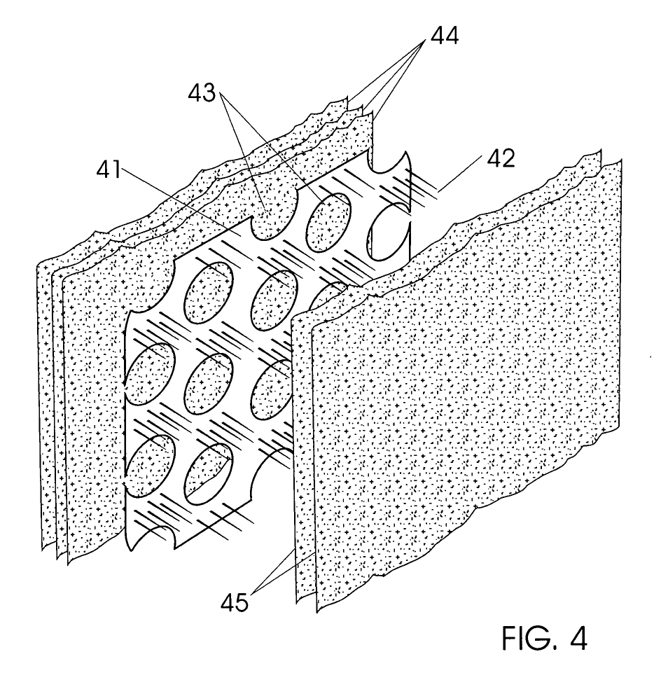

One important aspect of these blood sample collection systems relates to the manner in which microstructures are held perpendicular to the primary surface of these devices. FIG. 4 is a perspective drawing of a plurality of layers or sheets which operate together. A substrate 41 such as a silicon wafer provides a rigid foundation upon which a plurality of microstructures 42 may be formed. The substrate may further include large holes 43 distributed evenly about. These holes provide ready transmission of fluids with respect to the two sides of the substrate. Cloth, fabric, or nonwoven material sheets 44 together form a first portion of the absorbent pad. Additional layers 45 on the other side of the substrate also form a portion of the absorbent pad. When all of the layers including the silicon wafer are brought together to form the pad, the substrate becomes buried therewithin. While the substrate is no longer exposed nor visible, the microstructure tips pass through the layers of one side and remain exposed. Accordingly, the absorbent pad is said to have integrated therewith the structural support (substrate) for the microstructures.

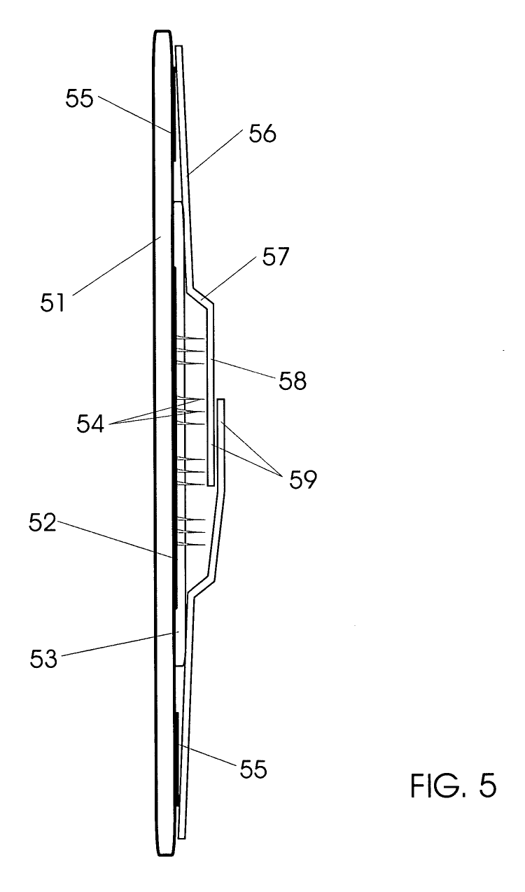

FIG. 5 is a side view of one version of these blood collection systems with a special protective cover element particularly illustrated. A main body strip 51 formed of plastic in example, forms basis upon which a substrate 52 may be directly affixed. An absorbent pad 53 is placed thereover the substrate in a manner whereby microstructures 54 may extend through the pad having their tips exposed. In this version, the substrate is directly affixed to the plastic strip rather than integrated with the absorbent pad.

The main body strip has thereon adhesive material 55 at either end. In this way, one may apply the device to the skin surface such that the microstructures and absorbent pad are in contact with and well coupled to the skin. While the primary function of the adhesive material is coupling to a user's skin, a secondary function also includes one in which a protective cover is temporarily affixed to the device.

Protective cover 56 may be provided as a hard plastic or semi-hard plastic cover member which operates to protect the device prior to its use. The protective cover may include a spatial accommodation for the microstructures whereby the cover provides a significant volume of space in which those microstructures occupy. To account for this, some versions of these protective covers include a shaped cover element. Rather than a flat sheet, these protective cover elements may include a ‘knee-bend’ portion 57 and second flat portion 58. When the protective cover it is held to the main body strip via the adhesive, the knee-bend and second flat portion provide for a cavity of space in which the microstructures reside.

In some preferred versions, a protective cover is formed of two cooperating element portions. These may form an overlap 59 as shown. The protective cover is easily released from the adhesive as it is removed prior to being applied to a user/donor's skin surface. Once a protective cover is removed and discarded, microstructures are exposed and may be applied to a user's skin surface.

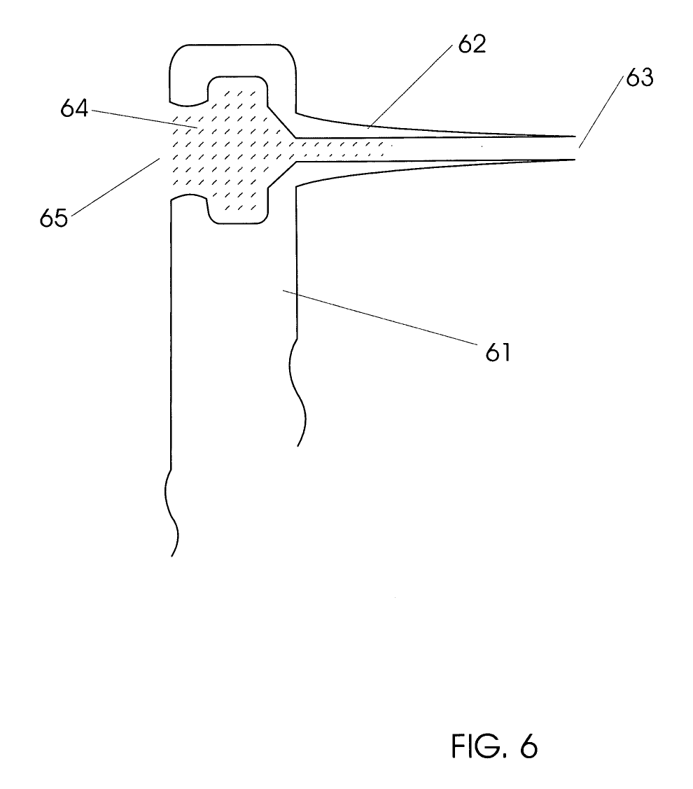

In certain special versions, an advanced microstructure is formed with a view to improving functionality of the entire system. In a manner similar to that in which a mosquito prepares his host with a blood thinner, these systems can be arranged with special-purpose chemistry for facilitating blood draw. A specialized substrate 61 is shown in FIG. 6 with a hypodermic needle type microstructure 62. An orifice 63 provides means by which a chemical blood thinner 64 stored in a substrate cavity or reservoir 65 may be transferred from the device to the donor. Without need for mechanisms to inject the chemical, natural fluid migration of blood tends to cause the agent to mix with and thin blood at the orifice. The blood and thinner will further mix at the injection site and ultimately cause blood to more easily be drawn into and towards the absorbent pad.

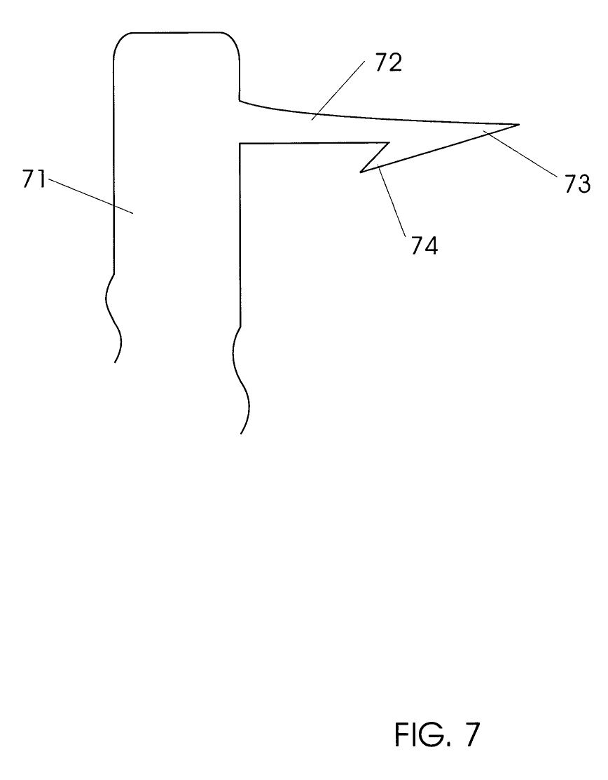

Another important type of microstructure relates to one which further facilitates penetration of the device in a user's tissue to which it is applied, and also mimics nature. A ‘barb’ type microstructure may be formed onto a suitable substrate via crystal growth, microlithography, and etching processes whereby the barb operates to pass through tissue in a preferred direction. Since these devices are merely applied to the surface of the skin, without typical mechanical means of causing injection, they rely upon the gentle pressure which occurs with light contact. To improve the tendency of the microstructure to get deep enough into the tissue to reach blood, a barb shaped microstructure is an improvement in some versions. FIG. 7 illustrates. A silicon substrate 71 has grown thereon a crystal barb 72 element with tip 73 and hook 74. While the illustration shows but a single barb microstructure, it will be understood that many, and even many tens of barbs may be provided about a substrate surface to effect a system capable of penetrating tissue and drawing therefrom a blood sample which may be absorbed into the cooperating pad.

While the core element described herein most critically includes the apparatus or device which is affixed to a donor skin for collecting blood, an important optional aspect of the invention includes the apparatus as well as a supporting kit. In some preferred embodiments, an easy-to-use, direct-to-consumer blood collection kit includes a blood collection apparatus as a primary element. In addition, these kits include a shipping box 81 suitable for return shipping delivery via government postal services or private delivery services such as Federal Express or UPS between a user donor and a laboratory including support for a user effected label (addressee) change.

Further, these direct-to-consumer blood collection kits include means for supporting items to be shipped in distinct receptacles. A foam core element 82 having an external surface symmetric in size and shape to the shipping box additionally comprises two distinct receiving cavities including: a slot receiving cavity 83 for receiving and holding therein and unused fresh blood collection apparatus 84 contained in a protective sterile disposable envelope 85. In addition, the foam core element includes a receiving cavity 86 to accommodate a plastic return container 87 which may include therein a blood stabilization and preserving fluid. Additionally, the container may be provided with a liquid tight seal suitable for safe containment of medical and biological matter. Further, the kit may be further comprised of an instruction card or pamphlet 88 including text and/or graphics to illustrate proper uses. Finally, the kit may also include an easy to use pre-addressed label 89 which permits the addressee to be changed on the two-way shipping box for the return trip. It is a primary function of these kits to permit those with little or no training whatever to easily get a blood sample into a safe container for return to the laboratory.

One will now fully appreciate how advanced easy-to-use blood sample collection apparatus and kits which support the direct-to-consumer need may be realized. Although the present invention has been described in considerable detail with clear and concise language and with reference to certain preferred versions thereof including best modes anticipated by the inventors, other versions are possible. Therefore, the spirit and scope of the invention should not be limited by the description of the preferred versions contained therein, but rather by the claims appended hereto.

What is claimed is:

1) Blood sample collection apparatus comprising:
a piercing element;
an absorbent pad; and
an adhesive strip,
said piercing element is affixed to said absorbent pad with at least one piercing microstructure extending substantially orthogonally therefrom,
said absorbent pad is affixed to one side of said adhesive strip, the strip having adhesive material on either side of the absorbent pad.

2) Blood sample collection apparatus of claim 1, said absorbent pad is comprised of a plurality of layers and said piercing element is integrated therewithin said absorbent pad layers.

3) Blood sample collection apparatus of claim 1, said piercing element includes a planar structure formed of a crystalline wafer.

4) Blood sample collection apparatus of claim 3, said microstructure is formed in a crystal growing process.

5) Blood sample collection apparatus of claim 3, said microstructure is a wafer with a plurality of holes provided therein.

6) Blood sample collection apparatus of claim 3, said at least one piercing microstructure is further characterized as either from the group: lances, barbs, needles, pins, knives, spikes.

7) Blood sample collection apparatus of claim 6, said at least one piercing element is further characterized as a hypodermic needle.

8) Blood sample collection apparatus of claim 7, said needle is formed integrally with a substrate having a reservoir therein, said reservoir containing a blood thinning or anticoagulant agent.

9) Blood sample collection apparatus of claim 6, said at least one piercing element is further characterized as a barb.

10) Blood sample collection apparatus of claim 1, said absorbent pad further comprises integrated therewithin at least one chemical agent.

11) Blood sample collection apparatus of claim 10, said at least one chemical agent is either from the group including: numbing agents, blood anticoagulants, blood anticlotting agents, blood thinners, antibacterial agents, perseverant, and blood stabilizers.

12) Blood sample collection apparatus of claim 11, said at least one chemical agent includes a plurality of agents where either agent is spatially removed from either other.

13) Blood sample collection apparatus of claim 12, further comprises a numbing agent disposed nearest an exposed surface of said absorbent pad.

14) Blood sample collection apparatus of claim 1, further comprises a indicator system arranged in opposition to said piercing element, said indicator system is responsive to the presence of a sufficient quantity of blood at the absorbent pad, the indicator system arranged to provide visible indicia when blood is present.

15) Blood sample collection apparatus of claim 14, said indicator system is based upon a chemical state change in response to presence of matter of a prescribed pH.

16) Blood sample collection apparatus of claim 15, said indicator system includes shaped indicia of a ‘positive’ or ‘affirmative’ nature such as a ‘plus’ sign or ‘check’ symbol.

17) Blood sample collection apparatus of claim 14, said indicator system is comprised of a cover element having a window therein, a medium which changes state in the presence of blood.

18) Blood sample collection apparatus of claim 17, said cover element and change medium is disposed on a side of said adhesive strip opposite to the side in which the absorbent pad is affixed, the adhesive strip further having via holes provided therein whereby blood in the absorbent pad may migrate to the change medium.

19) Blood sample collection apparatus of claim 1, further comprising a removable protective cover element arranged to be temporarily affixed via the strip adhesive and further to cover and protect the apparatus microstructures.

20) Blood sample collection apparatus of claim 19, said cover strips further comprise a ‘kneebend’ discontinuity to account for and create a cavity between the cover and adhesive in which the microstructures may be accommodated therein.

21) Blood sample collection kits comprising:
a durable outer container;
a foam core;
a blood sample collection apparatus;
a sterile envelope; and
a return container,
said durable outer container is suitable for two-way transmission in the mails or private delivery services further having an outer surface suitable for accommodating addressee information which may be changed,
said foam core substantially similar but slightly smaller in shape and size whereby the foam core fits snugly within the outer container, the foam core further having at least two voids or slots formed therein to accommodate said envelope and return container.

22) Blood sample collection apparatus of claim 21, said blood sample collection apparatus is a blood sample collection apparatus comprising: a piercing element; an absorbent pad; and an adhesive strip, said piercing element is affixed to said absorbent pad with at least one piercing microstructure extending substantially orthogonally therefrom, said absorbent pad is affixed to one side of said adhesive strip, the strip having adhesive material on either side of the absorbent pad.

23) Blood sample collection kits of claim 21, further comprising a preprinted return address label and system use instruction set.
